(19) United States
(12) Patent Application Publication
(10) Pub. No.: US 2006/0206120 A1
(43) Pub. Date: Sep. 14, 2006

(54) Nose clip

(75) Inventor: Burrell E. Clawson, Newport Beach, CA (US)

Correspondence Address: STOUT, UXA, BUYAN & MULLINS LLP, 4 VENTURE, SUITE 300, IRVINE, CA, 92618, US

(73) Assignee: Enternet Medical, Inc., Las Vegas, NV (US)

(21) Appl. No.: 11/075,188

(22) Filed: Mar. 8, 2005

(51) Int. Cl. A61B 17/08

(52) U.S. Cl. 606/151

(57) ABSTRACT

A nose clip is provided having a clip portion including two curved elongated arms and a curved bridge portion therebetween. The arms and bridge portion advantageously form a substantially C-shaped structure. Each of the arms includes a flexible joint region and a pad connected thereto. The joint region is structured so as to enable a wide degree of movement of the pad relative to another portion of the arm. The pads preferably have non-circular nose contact surfaces, for example, the nose contact surfaces of the pads may be substantially oval in shape. The nose clip includes two lever portions projecting from the clip portion which are structured such that moving the two lever portions toward one another causes the pads to be drawn apart from one another, for example, by a distance sufficient to facilitate placement of the nose clip on a nose of a human or animal. In one embodiment, nose clip is substantially entirely molded from a single material as a unitary structure and requires no assembly of parts.

This invention generally relates to nose clips, and more particularly relates to nose clips that are temporarily clipped onto human or animal noses during medical procedures, medical tests, and the like.

### BACKGROUND OF THE INVENTION

Pulmonary function tests generally include a set of diagnostic tests designed to help evaluate how the entire respiratory system is functioning. For example, diffusion capacity tests help measure the efficacy (or lack thereof) of the lungs in transferring oxygen from inhaled air into the bloodstream. Spirometry generally measures how well air is exhaled from the lungs, and lung volume measurement tests help establish the volumetric capacity of the lungs by measuring the quality of air inhaled/exhaled during a breathing cycle. These tests help diagnose a host of restrictive respiratory disorders such as asthma, chronic obstructive pulmonary disease and the like, as well as various obstructive and interstitial lung diseases. Nose clips are used during these and other medical tests and procedures where it is desirable to restrict a person's breathing through the nose, so all respiration goes through the flow sensor.

There are presently only a few nose clip designs available that are specifically marketed to physicians who perform these tests and procedures. Such conventional nose clips are made to be disposable after one use, and thus are made from inexpensive molded materials. Unfortunately, these conventional nose clips are often uncomfortable to wear, even for a short time period, and/or are not sufficiently effective in restricting breathing through the nose.

There continues to be a need for functionally better nose clips.

### SUMMARY OF THE INVENTION

New nose clips have been discovered. The present nose clips, for example, for medical diagnostic procedures and the like, are easy and inexpensive to manufacture, require no assembly, are comfortable and effective to use and suitable for use on noses of nearly all sizes and shapes. The present nose clips overcome many of the problems of conventional nose clips by providing a simple yet highly effective design that is disposable, inexpensive to manufacture, yet quite comfortable relative to conventional nose clips made for these purposes.

Generally, a nose clip in accordance with the present invention comprises a clip portion that gently clips to a nose to close the nostrils thereof. The clip portion includes two arms, each arm including a pad movable relative to another portion of the arm, and a bridge region, for example a flexible bridge region intermediate and integral with the arms. The arms and bridge portion are preferably all curved in substantially the same direction, thereby forming a substantially C-shaped structure.

Each pad include a surface that contacts a nose when the nose clip is placed on the nose. In one advantageous embodiment, the surface has a non-circular perimeter. For example, the surface of the pad which contacts the nose has a substantially oval perimeter.

Each arm preferably comprises an elongated region and a flexible joint region connecting the pad to the elongated region. Advantageously, the joint region is structured so as to enable movement of the pad with respect to the elongated region. In a preferred embodiment, the joint region is effective in enabling the pad to be swiveled, pivoted and/or otherwise rotated to a desired position with an effect much like that of a universal ball joint in a mechanical mechanism. The oval shaped perimeter of the pad contact surface and the ability to move the pad into a desired position enables a custom fit of the nose clip to a wide variety of noses. The contact surface of the pads may include textured surface regions for facilitating the securement of the nose clip on the nose in the desired position.

The nose clip further includes two lever portions, for example curved lever portions, projecting from the clip portion and spaced apart from one another by the bridge region. The nose clip is structured so that manipulation of the two lever portions toward one another causes the pads of the arms to be separated by a distance sufficient to facilitate placement of the nose clip on a nose of a human or animal. The nose clip may further comprise a pair of reinforcement elements disposed between the clip portion and a different one of the lever portions. To accommodate a particularly wide nose, it is possible for the clinician to ‘stretch-open’ the pads so they are wider apart by applying enough force to permanently deform or stretch the C-shaped structure. The present clips are ‘flexibly force limiting’ in that the structure of the clips substantially prevents a clinician from over-opening and permanently deforming the clip when opened to accommodate an average sized nose.

In an especially advantageous embodiment of the invention, the nose clip is substantially entirely molded from a single material, for example a moldable plastic material, as a unitary structure. Preferably the nose clips of the invention are formed as unitary in structure.

Each and every feature described herein, and each and every combination of two or more of such features, is included within the scope of the present invention provided that the features included in such combinations are not mutually inconsistent.

### BRIEF DESCRIPTION OF THE DRAWINGS

The present invention will be more clearly understood and the advantages thereof more readily appreciated with reference to the following detailed description and claims when considered in conjunction with the accompanying drawings.

FIGS. 1a and 1b are perspective views of two different PRIOR ART nose clips commonly used during certain medical tests and procedures.

FIG. 2 is top plan view of an improved nose clip in accordance with the present invention.

FIGS. 3 and 4 are side views of the nose clip shown in FIG. 2, showing a pad thereof disposed in two different positions for accommodating two differently shaped noses.

FIG. 5 is a perspective view of the nose clip shown in FIGS. 2-4, illustrating the versatility of positioning of the nose pads with respect to the balance of the nose clip.

FIG. 6 is a top view of a further embodiment of the invention.

### DETAILED DESCRIPTION

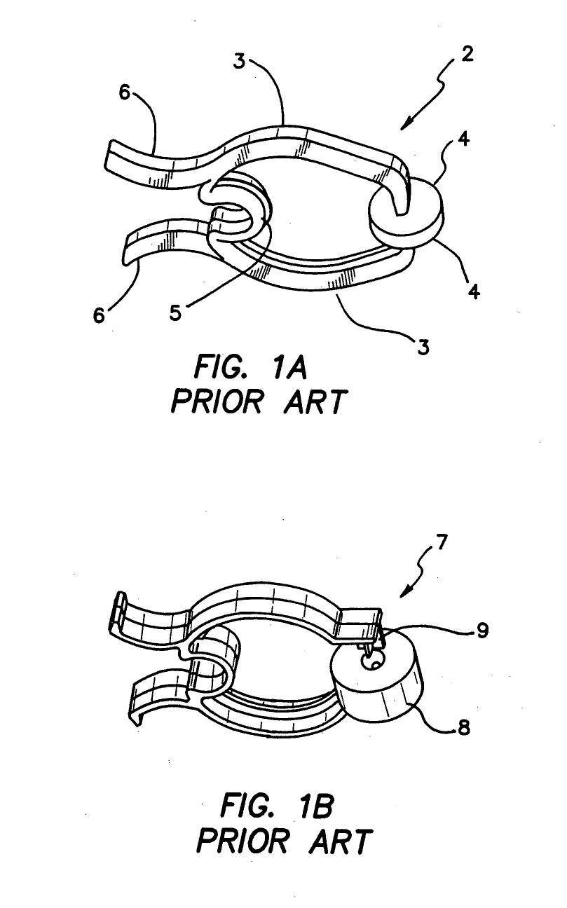

Turning now to FIG. 1a common PRIOR ART nose clip is shown generally at 2. Nose clip 2 includes elongated regions 3 with circular members 4 immovably fixed with respect to the portion of the elongated region of which they are attached. This nose clip includes a markedly inwardly curved region 5 connecting the two elongated regions 3. The nose clip 2 is secured to a nose by a user pinching levers 6 toward one another, which causes inwardly curved region 5 to bend inwardly, and thereby pulling the elongated regions apart which results in disc shaped members 4 pulling away from one another. The nose clip 2 is then placed on the nose and allowed to spring back into its unpinched configuration leaving the members 4 generally covering and pressing inwardly against the nostrils.

FIG. 1b shows another PRIOR ART nose clip generally at 7. This nose clip 7 is substantially the same as nose clip 2, except that nose clip 7 includes soft foam pads 8 (only one is shown) removably coupled to connectors 9 for providing comfort to a wearer. Unfortunately, although nose clip 7 may be considered more comfortable to wear than nose clip 2, the foam pads 8 may prevent the nose clip 7 from achieving the closing force necessary to ensure no respiration through the nostrils.

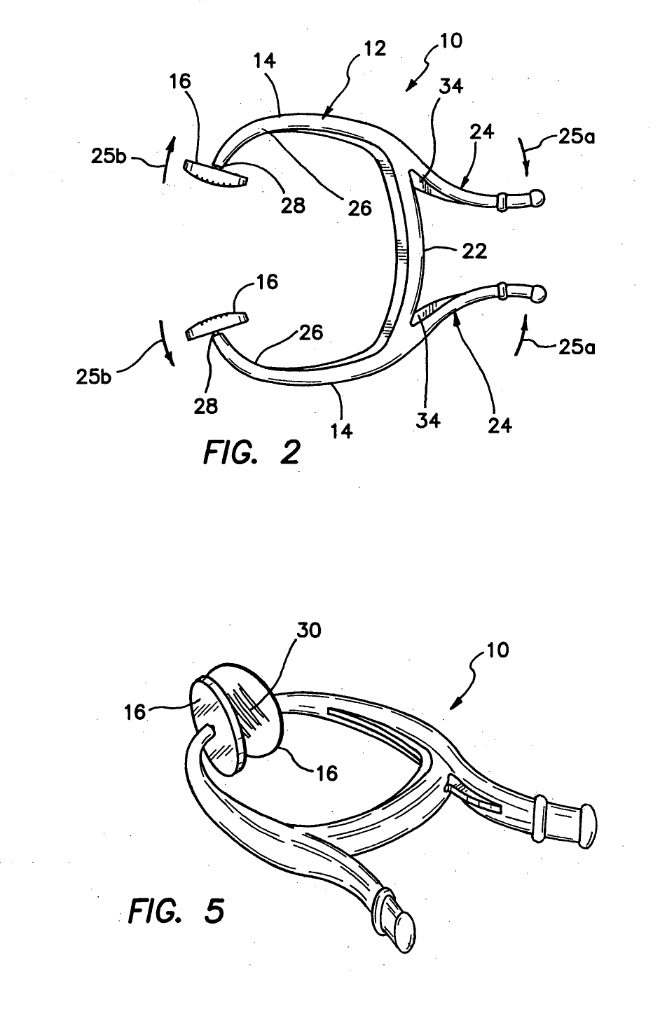

Turning now to FIG. 2 an improved nose clip in accordance with the present invention is shown generally at 10. The nose clip 10 includes a clip portion 12 including two arms 14, and a bridge region 22 intermediate and connecting the two arms 14. Each arm 14 of the nose clip 10 includes a pad 16 which is movably connected to another portion of the arm 14. The nose clip 10 further includes two lever portions 24 projecting outwardly from the clip portion 12 and spaced apart from one another by the bridge region 22. Each of the arms 14, bridge region 22 and lever portions 24 are curved, elongated structures. The nose clip 10 is structured such that manipulation of the two lever portions 24 (e.g. in the direction indicated by arrows 25a) causes the pads 16 of the arms 14 or separated from one another an amount sufficient to facilitate placement of the nose clip 10 on a nose of a human or animal.

The arms 14 and bridge region 22 are curved generally along a common arc, thereby defining a substantially C-shaped structure as shown. Advantageously, the bridge region 22 does not generally bend inwardly as does comparable structure in prior art nose clips (for example inwardly curving structure 5 of PRIOR ART nose clip 2). Manipulation, for example pinching, of the lever portions 24 toward one another (in the direction indicated by arrows 25a) causes the pads to be moved apart (e.g. in the direction indicated by arrows 25b) and separated. In addition, the outwardly curving bridge region 22 provides the present nose clip 10 with substantial open space between the arms 14 to comfortably accommodate a relatively large nose, compared to a similarly sized prior art nose clip.

Preferably, nose clip 10 is formed of a single material, for example, the nose clip 10 is molded as a unitary structure made of a plastic material, for example polypropylene. Therefore, the nose clip 10 requires no assembly of parts and can be manufactured quite quickly and inexpensively.

Still referring to FIG. 2, the arm 14 comprises an elongated region 26, a flexible joint region 28 and the pad 16. It is noted that the pad 16, flexible region 28 and elongated region 26 are all integrally formed or molded components.

The structure of the nose clip 10 enables the clip 10 to be useful for comfortably and effectively closing noses of a variety of shapes and sizes. For example, the joint region 28 is structured to permits the pad 16 to resiliently swivel, tilt and/or rotate with respect to the elongated region 26.

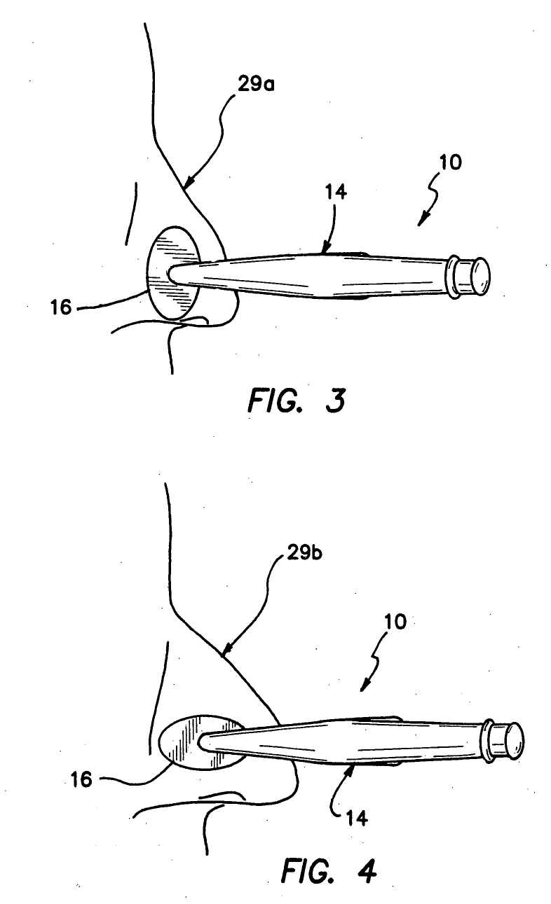

Turning now to FIGS. 3 and 4, the nose clip 10 is shown as being used to close different nose types. More particularly, nose clip 10 shown in FIG. 3 as being used to close a smaller sized, narrow nose 29a. In contrast, the same nose clip 10 shown in FIG. 4 is being used to close a relatively broad nose 29b.

An advantage of the nose clip 10 is a preferred generally non-circular shape of the perimeter of a contact surface of the nose pad 16. More specifically, each pad 16 is shown to have a surface 30 that contacts a nose wherein the contact surface 30 has a non-circular perimeter, for example the contact surface 30 has a substantially oval perimeter or other elongated shape. This structure allows the nose clip 10 to be effective in comfortably closing noses of various shapes and sizes, for example, enabling a wearer to selectively position the major axis and/or minor axis of the elongated pad 16 to achieve the best fit. It can be appreciated from FIGS. 3 and 4 that pads 16 are positioned differently on each differently sized and/or shaped nose.

As shown in FIG. 5, the freedom of movement of the pads 16 on the joint region 28 and the oval shape of the pads 16 allows the nose clip to be custom fit to substantially any nose size and shape. The pads 16 can be positioned to match the most varied nose sizes and shapes of the wearers when applied thereto. Pads 16 are pivotable in all directions with respect to joint member 28 such that the nose clip 10 will comfortably fit substantially any nose in an effective and comfortable manner. FIG. 5 shows the nose pads 16 swiveled toward each other near a forward region of the nose clip 10 and away from each other near a rearward region of the nose clip 10.

Turning now to FIGS. 2 and 5, at least a portion of the contact surface 30 of the pads may comprise a textured surface that facilitates securement of the nose clip 10 to the nose, for example to prevent the nose clip 10 from sliding off the nose during use. Once placed on the nose, the pads 16 remain substantially fixed in place with respect to the nose until the nose clip 10 is intentionally removed therefrom, for example, by a user pinching together the lever portions 24 to draw the pads 16 apart from one another (see FIG. 2). The nose clip 10 may further include a pair of reinforcement elements 34, each reinforcement element being disposed between the clip portion 12 and a different one of the lever portions 24.

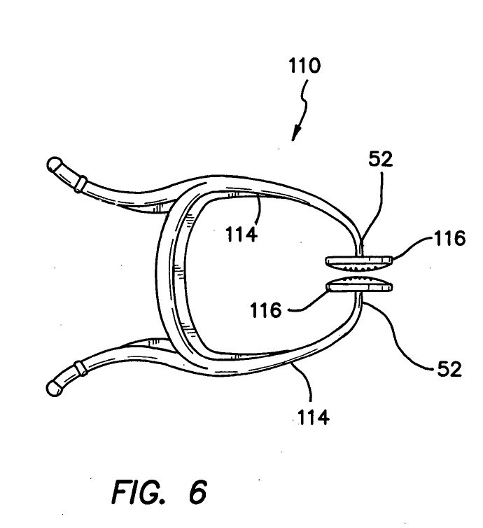

Turning now to FIG. 6, a nose clip 110 in accordance with a further embodiment of the invention is shown. Except as expressly described herein, nose clip 110 is the same as nose clip 10 and features of nose clip 110 which correspond to features of nose clip 10 are designated by the corresponding reference numerals increased by 100.

The major distinction between nose clip 110 and nose clip 10 is the structure of the region between each arm 114 and pad 114. Unlike nose clip 10 which includes the relatively distinct joint region 48, nose clip 110 includes arms 114 having tapered distal portions 52 that integrally connect to pads 116 without a distinct joint region. Like nose clip 10, nose clip 110 may be, and preferably is, a molded unitary structure. Distal portions 52 are sufficiently flexible to allow pads 116 to pivot as described and shown elsewhere herein.

While this invention has been described with respect to various specific examples and embodiments, it is to be understood that the invention is not limited thereto and that it can be variously practiced within the scope of the following claims.

What is claimed is:

1. A nose clip comprising:
a clip portion including two arms, each arm having a pad movable relative to another portion of the arm and a bridge region connecting the two arms; and
two lever portions projecting from the clip portion and spaced apart from one another by the bridge region;
the nose clip being formed of a single material and being structured such that manipulation of the two lever portions causes the pads of the arms to be separated by a distance sufficient to facilitate placement of the nose clip on a nose of a human or animal.

2. The nose clip of claim 1 wherein each arm has pad pivotally movable relative to another portion of the arm.

3. The nose clip of claim 1 formed as a unitary structure.

4. The nose clip of claim 1 wherein the arms and bridge region have a substantially C-shaped structure.

5. The nose clip of claim 1 wherein the bridge region is structured so that manipulation of the two lever portions toward one another causes the bridge region to bend in a direction away from the pads.

6. The nose clip of claim 1 further comprising a pair of reinforcement elements each reinforcement element disposed between the clip portion and a different one of the lever portions.

7. The nose clip of claim 1 wherein the pads include surfaces that contact a nose when the nose clip is placed on the nose.

8. The nose clip of claim 7 wherein the surfaces have non-circular perimeters.

9. The nose clip of claim 7 wherein the surfaces have substantially oval perimeters.

10. A nose clip comprising:
a substantially C-shaped clip portion including two arms each having a pad which is movable relative to another portion of the arm, and a bridge region connecting the two arms, the arms having complementary curved shapes; and
two lever portions projecting from the clip portion and spaced apart from one another by the bridge region;
the nose clip being structured such that manipulation of the two lever portions toward one another causes the pads of the arms to be moved apart from one another by a distance sufficient to facilitate placement of the nose clip on a nose of a human or animal.

11. The nose clip of claim 10 wherein the clip portion and the lever portions and formed of a single material.

12. The nose clip of claim 10 formed as a unitary structure.

13. The nose clip of claim 10 wherein each pad is pivotally movable relative to the portion of the arm of which it is a part.

14. The nose clip of claim 10 further comprising a pair of reinforcement elements each reinforcement element disposed between the clip portion and a different one of the lever portions.

15. The nose clip of claim 10 wherein the pads include surfaces that contact a nose when the nose clip is placed on the nose.

16. The nose clip of claim 15 wherein the surfaces have non-circular perimeters.

17. The nose clip of claim 15 wherein the surfaces have substantially oval perimeters.

18. A nose clip comprising:
a substantially C-shaped clip portion comprising:
two arms, each arm having a curved elongated region, a pad and a flexible joint region connecting the pad to the elongated region, each joint region being structured so as to enable movement of the pad with respect to the elongated region, each pad including a contact surface having a substantial oval perimeter, and
a curved bridge region joined to each of the arms at another end of the elongated region of each arm;
two curved lever portions, each lever portion joined to and projecting from the other end of the elongated region of one of the a different arms; and
a pair of reinforcement elements, each reinforcement element disposed between the clip portion and a different one of the lever portions,
the nose clip being structured such that manipulation of the two lever portions toward one another causes the pads to be moved apart from one another by a distance sufficient to facilitate placement of the nose clip on a nose of a human or animal, and the nose clip being substantially entirely molded from a single material as a unitary structure.
